(19) United States
(12) Patent Application Publication
(10) Pub. No.: US 2026/0351412 A1
(43) Pub. Date: Nov. 5, 2026

(54) APPLYING MACHINE LEARNING MODEL COMBINATIONS FOR MEDICAL DIAGNOSIS

(71) Applicant: Aledade, Inc., Bethesda, MD (US)

(72) Inventors: Farzad Mostashari, Bethesda, MD (US); Denys Liubyvyi, Kensington, MD (US); William Woolston, New Haven, CT (US); Yushi Homma, Rancho Palos Verdes, CA (US); Ritwik Tewari, Los Altos, CA (US); Siraj Farage, San Diego, CA (US); Joseph Neumann, Fitchburg, WI (US); Anasuya Das, Demarest, NJ (US); Jason Keith King, Nashville, TN (US); Jacqueline Kathleen Kueper, Brooklyn, NY (US); Michael Gah-ming Fanton, Chicago, IL (US); Julio Cárdenas Rodríguez, Catalina, AZ (US)

(21) Appl. No.: 19/663,382

(22) Filed: Apr. 30, 2026

Related U.S. Application Data
(60) Provisional application No. 63/798,953, filed on May 2, 2025.

(51) Int. Cl. G16H 50/20; G16H 10/60; G16H 50/70; G16H 70/20

(52) U.S. Cl. CPC G16H 50/20; G16H 10/60; G16H 50/70; G16H 70/20

(57) ABSTRACT

Aspects of the inventive concepts involve a system and a method for identifying a medical diagnosis, comprising: receiving, by a first model, structured data, wherein the first model includes a Machine Learning (ML) model; generating, by the first model including the ML model, a first set of potential diagnoses; converting the structured data into a set of semi-structured data; and processing, by a second model, the set of semi-structured data to generate a second set of potential diagnoses that is a subset of the first set of potential diagnoses. The second model includes a Large Language Model (LLM). The second set of potential diagnoses is smaller than the first set of potential diagnoses.

### RELATED APPLICATIONS

This application claims priority to U.S. provisional patent application No. 63/798,953 filed May 2, 2025 and titled “APPLYING MACHINE LEARNING MODEL COMBINATIONS FOR MEDICAL DIAGNOSIS,” the contents of which are incorporated by reference in their entirety.

### FIELD OF INTEREST

The present inventive concepts relate to systems and methods useful for interpreting electronic records systems, and more specifically, a two-stage system combining Machine Learning (ML) and Large Language Models (LLMs) to identify and verify potential diagnoses, from electronic health record (EHR) data.

### BACKGROUND

Healthcare Hierarchical Condition Category (HCC) Codes are part of a risk adjustment model used in Medicare and other insurance programs to determine how Medicare reimburses healthcare providers such as doctors and hospitals, which may be grouped into Accountable Care Organizations (ACOs). Healthcare providers typically maintain databases of its patients' medical histories, referred to as Electronic Health Record (EHR) systems, which store patient information related to medical care provided by the practice. Software systems integrated with EHR can help streamline the process of highlighting potential HCC codes by integrating clinical data and diagnosis information directly into the provider's workflow. HCC codes are grouped hierarchically, where a more severe condition can be assigned to a higher-level HCC code, reflecting its greater healthcare cost burden. These codes can range from chronic conditions like diabetes, heart disease, or chronic kidney disease, to more complex conditions like cancer, stroke, or organ failure. The HCC codes can therefore be used to calculate a risk score for each patient, which is used by Medicare to adjust payments to healthcare providers. The higher the risk score, the higher the expected healthcare costs, and the higher the reimbursement.

Artificial Intelligence (AI) plays a growing role in automating and improving the accuracy of HCC coding. For example, AI can be implemented to analyze large volumes of medical records, extracting suspected diagnoses that are later linked to appropriate HCC codes. AI can also identify patterns related to chronic diseases like diabetes, cardiovascular disease, and other conditions commonly associated with HCC codes. Machine Learning (ML) models can be trained on historical coding data to predict the most likely HCC code based on patient records. These models can continuously improve over time as they process more data. With regulatory requirements becoming stricter, AI can help healthcare providers remain compliant with HCC coding rules by flagging potential coding mistakes or missed diagnoses before submission. For example, a fixed heuristic rule may establish that all users of a particular medication are expected to have a particular medical condition without recognizing that the medication may be used for a different medical condition. Besides, heuristic rules have issues with respect to a limited coverage of potential diagnoses, referred to as suspects, and may have low sensitivity resulting in missed diagnoses, which is a problem when the goal is to improve patient care by identifying as many true cases as possible, particularly chronic diseases.

When evaluating the ML models, performance is paramount. Sensitivity and PPV (positive predictive value, also known as precision), are metrics used to evaluate the performance of a predictive classification model. Sensitivity, also known as recall, is used to evaluate machine model performance because it allows one to see how many positive instances the model was able to correctly identify as compared to total positives. PPV, on the other hand, is used to measure the proportion of true positives that are correctly identified by the model. A low precision indicates that a model is generating an excessive number of false positives. Conventional models are challenged with balancing sensitivity and precision. For example, when training a model to analyze data regarding a patient having a disease, a reduction in a decision threshold will result in more patients classified as having the disease, resulting in an increase in sensitivity but decrease in precision. However, an increase in the decision threshold may result in the model classifying fewer patients as diseased, which typically increases PPV but also increases false negatives, resulting in a decrease in sensitivity.

An additional challenge in medical AI systems involves retrieving relevant clinical evidence from heterogeneous medical data sources. Healthcare data typically exists in two distinct forms: structured EHR data (such as lab results, medications, and diagnosis codes) and unstructured data, for example, clinical notes such as provider documentation and clinical narratives. Current systems may struggle to create data artifacts that are AI-native and that effectively combine information from both data types, which can lead to incomplete context for AI models. While processing of unstructured data has been more extensively documented in this area, the creation of LLM-optimized semi-structured data that bridges both structured and unstructured sources represents a novel approach. This challenge highlights the need for retrieval architectures that can effectively access and combine clinical evidence from multiple heterogeneous data streams.

It is desirable for a machine learning model used in medical record processing to provide an acceptable tradeoff between sensitivity and precision, ideally maximizing both metrics.

### SUMMARY

In accordance with aspects of the inventive concepts, provided is a method for identifying a medical diagnosis. In the method, a first model including a Machine Learning (ML) model receives structured data and generates a first set of potential diagnoses. The structured data is converted into a set of semi-structured data. A second model processes the set of semi-structured data to generate/refine a second set of potential diagnoses. The second model is an LLM. The second set of potential diagnoses is smaller than the first set of potential diagnoses.

In another aspect, in a method for diagnosis code identification, a first model including a Machine Learning (ML) model receives a plurality of diagnosis codes in a structured format and grouped into categories that represent serious or chronic conditions. The first model generates a prediction of a first set of diagnosis codes for a patient. The diagnosis codes structured format is converted into a set of semi-structured data. A second model including an LLM processes the set of semi-structured data to determine a second set of diagnosis codes from the diagnosis codes.

In another aspect, a system for identifying and verifying a potential medical diagnosis comprises at least one processor and at least one computer storage device. A high sensitivity machine learning model is stored at the at least one computer storage device and executable by the at least one processor to: electronically access at least one electronic health records (EHR) system comprising a plurality of patient EHRs and identify a first set of suspect diagnoses using low decision thresholds. The system further comprises a converter to convert structured EHR data including the plurality of patient EHRs and the first set of suspect diagnoses into a cohesive unstructured patient narrative and a LLM stored at the at least one computer storage device and executable by the at least one processor to identify a second set of potential diagnoses from the first set of suspect diagnoses.

In another aspect, a method for identifying a medical diagnosis comprises receiving, by a first model including a Machine Learning (ML) model, structured data, generating, by the first model, a first set of potential diagnoses, converting the structured data into a set of semi-structured data, and processing, by a second model including a Large Language Model (LLM), the set of semi-structured data to generate a second set of potential diagnoses that is a subset of and smaller than the first set of potential diagnoses.

In another aspect, a method for diagnosis code identification comprises receiving, by a first model including a Machine Learning (ML) model, a plurality of diagnosis codes in a structured format and grouped into categories that represent various medical conditions and events, generating, by the ML model, a prediction of a first set of diagnosis codes for a patient, converting the diagnosis codes structured format into a set of semi-structured data, and processing, by a second model including an LLM, the set of semi-structured data to determine a second set of diagnosis codes from the diagnosis codes.

In another aspect, a system for identifying and verifying a potential medical diagnosis comprises at least one processor and at least one computer storage device, a high sensitivity machine learning model stored at the at least one computer storage device and executable by the at least one processor to electronically access at least one electronic health records (EHR) system comprising a plurality of patient EHRs and identify a first set of suspect diagnoses using low decision thresholds, a converter to convert structured EHR data including the plurality of patient EHRs and the first set of suspect diagnoses into a cohesive unstructured patient narrative, and a LLM stored at the at least one computer storage device and executable by the at least one processor to identify a second set of potential diagnoses from the first set of suspect diagnoses.

In another aspect, a method for clinical reasoning using parallel semantic search comprises performing parallel retrieval from two distinct data streams comprising structured patient data and unstructured clinical notes, wherein the structured patient data includes labs, medications, and diagnoses, and the unstructured clinical notes include provider documentation, using separate query builders optimized for each data type, deduplicating and combining results from both data streams into unified context, and organizing the unified context as episodic snippets comprising encounter-level patient summaries that temporally organize both structured and unstructured data by clinical episode.

In another aspect, a method for verifying LLM-generated medical evidence comprises generating, by an LLM, evidence and supporting rationale for a potential medical diagnosis, systematically verifying each piece of evidence by cross-referencing against original patient record source text, determining whether each evidence item has verbatim or near-verbatim support in the source text, and flagging or removing evidence items that cannot be verified before presentation to clinicians.

In some aspects, the LLM uses clinical practice guidelines to structure both a prompt and an expected output format, and the LLM output is organized as checklist-aligned fields including a criteria header from the clinical practice guidelines, an answer, a source snippet as verbatim proof, and a confidence level.

In some aspects, when the LLM identifies incomplete evidence for a suspected diagnosis, the method further comprises generating guideline-based recommendations for next steps including specific lab tests to order, diagnostic procedures to perform, or specialist referrals, wherein the recommendations are derived from the same clinical practice guidelines used for diagnosis validation.

In some aspects, a method for retrieving clinical evidence from heterogeneous medical data sources, comprising: performing parallel semantic search over structured patient data and unstructured clinical notes using optimized query builders for each data type; deduplicating and combining results from both searches; and organizing the combined results as encounter-level episodic patient summaries that temporally co-locate structured and unstructured data by clinical encounter date.

In some aspects, a method for verifying LLM-generated medical evidence, comprising: receiving evidence claims from an LLM; for each claim, retrieving source text from patient records and determining whether the claim has verbatim or near-verbatim support using string matching or semantic similarity scoring; and filtering claims by removing or flagging those lacking source support before presenting to clinicians.

In some embodiments, the LLM verification operation uses evidence-based clinical practice guidelines to structure the LLM prompt and validate outputs, generating checklist-aligned evidence elements that correspond to guideline criteria and include: criteria header, answer (yes/no/partial), evidence type, date, source snippet, and confidence level.

In some embodiments, the method further comprises when diagnosis criteria are not fully met, generating an actionable clinical recommendation based on the clinical practice guideline, the recommendation comprising at least one of: a laboratory test to order, a diagnostic procedure to perform, or a specialist referral.

### BRIEF DESCRIPTION OF THE DRAWINGS

Aspects of the present inventive concepts will become more apparent in view of the attached drawings and accompanying detailed description. The embodiments depicted therein are provided by way of example, not by way of limitation, whereas like reference numerals refer to the same or similar elements. The drawings are not necessarily to scale, emphasis instead being placed upon illustrating aspects of the invention. In the drawings:

FIG. 1 is a block diagram of an embodiment of a system for identifying and verifying potential medical diagnoses, in accordance with aspects of the inventive concepts.

FIG. 2 is a flow diagram of a process for potential diagnosis verification, in accordance with aspects of the inventive concepts.

FIG. 3 is a flow diagram of a method for ML model training and inference to predict potential diagnoses, in accordance with aspects of the inventive concepts.

FIG. 4 is a flow diagram of an LLM suspect verification operation, in accordance with aspects of the inventive concepts.

FIG. 5 is a graph illustrating a quality-sensitivity comparison of techniques for capturing diagnosis (Dx) suspects.

FIG. 6 is a graph illustrating a sensitivity-precision tradeoff of different models and comparison of models.

### DETAILED DESCRIPTION OF EXEMPLARY EMBODIMENTS

Various aspects of the inventive concepts will be described more fully hereinafter with reference to the accompanying drawings, in which some exemplary embodiments are shown. Aspects of the present inventive concepts may, however, be embodied in many different forms and should not be construed as limited to the exemplary embodiments set forth herein.

It will be understood that, although the terms first, second, etc. are being used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

To the extent that functional features, operations, and/or steps are described herein, or otherwise understood to be included within various embodiments of the inventive concept, such functional features, operations, and/or steps can be embodied in functional blocks, units, modules, operations and/or methods. And to the extent that such functional blocks, units, modules, operations and/or methods include computer program code, such computer program code can be stored in a computer readable medium, e.g., such as non-transitory memory and media, that is executable by at least one computer processor.

In brief overview, embodiments of the present inventive concept provide a two-stage system and method that combine Machine Learning (ML) and Large Language Models (LLMs) to identify and verify potential medical diagnoses. In doing so, the system and method address and solve the sensitivity-precision tradeoff that hampers traditional machine learning approaches. In some embodiments, a first model is a high-sensitivity ML model that identifies a broad set of suspect diagnoses using a low decision threshold. A decision threshold is the point at which a model classifies a sample as positive or negative. By setting low thresholds, the model becomes more lenient in classifying a case as positive, even if the confidence level is not as high. For example, the ML model is configured to catch every possible case to ensure that few if any potential diagnoses are missed, notwithstanding the high probability of more false positives. A high sensitivity, or recall, machine learning model, means that the model is excellent at identifying the positive cases, which aligns with the desirable goal of setting low decision thresholds. In lowering the threshold, the decision boundary is adjusted to be more inclusive of positive predictions. In other words, the ML model is more likely to classify a case as positive, which increases the recall, or number of positive cases. This is particularly important in medical diagnostics where one does not want to miss any potential positive cases (even at the cost of some false positives).

However, in ML models, a high sensitivity can come at the expense of precision since a low precision can result in an increase in false positives, i.e., the proportion of predicted positives that are actually true positives.

LLMs, on the other hand, when used separately are prone to hallucination when processing insufficient or inaccurate input data and can therefore be unreliable, especially with sensitive data such as EHRs medical data.

Embodiments of the present inventive concept address the forgoing by providing an LLM at the output of an ML model, and providing an interface between the ML model and the LLM that permits the system to preserve full control of the EHR data, while also increasing both sensitivity and precision (and addressing the tradeoff issues with conventional ML models) and providing the relevant context that allows a user to identify suspect for potential diagnosis. This permits the system to verify the relevance and clinical validity of the suspect diagnoses produced by the ML model, and in doing so permits the LLM to complement the ML model to balance breadth and accuracy with respect to suspect diagnoses.

In some embodiments, the system may employ a dual-stream retrieval architecture that performs parallel retrieval from two distinct data streams: structured patient data (such as labs, medications, and diagnoses) and unstructured clinical notes (such as provider documentation). Results from both streams may be deduplicated and combined into unified context organized as episodic snippets, which are encounter-level patient summaries that temporally organize both structured and unstructured data by clinical episode.

In some embodiments, the system may include a hallucination fact-checking verification layer that cross-references LLM-generated evidence against original patient record source text. Evidence items that cannot be verified may be flagged or removed before presentation to clinicians, helping to prevent medical data hallucination in high-stakes clinical applications.

In some embodiments, the LLM validation layer may use clinical practice guidelines to structure both the prompt and the expected output format, ensuring that AI reasoning follows established medical criteria. When the LLM validation identifies incomplete evidence for a suspected diagnosis, the system may generate specific guideline-based recommendations for next steps, such as lab tests to order, diagnostic procedures to perform, or specialist referrals.

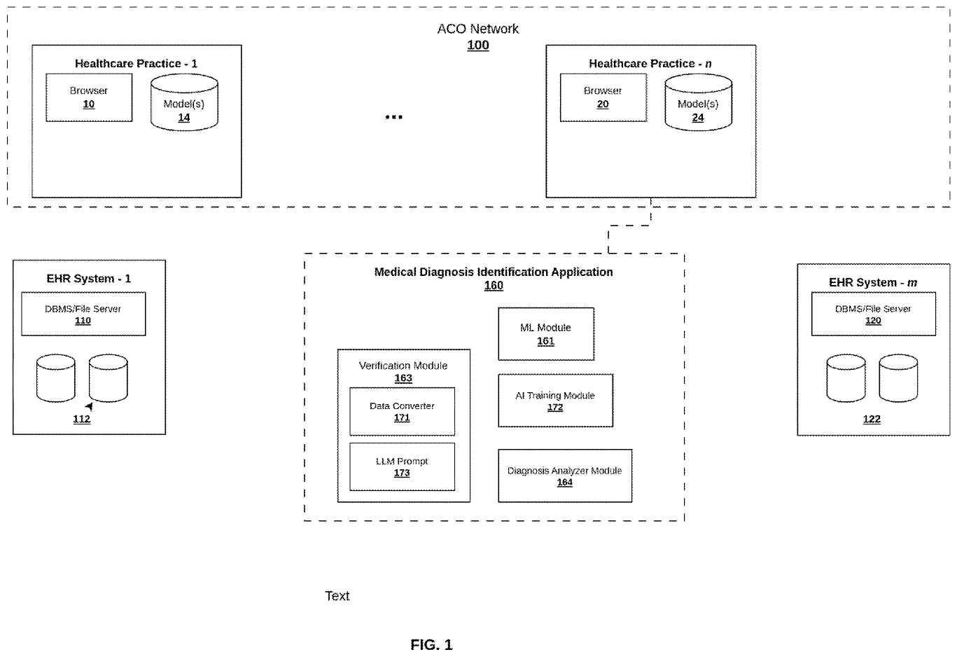

FIG. 1 provides an embodiment of a system for identifying and verifying potential medical diagnoses. In some embodiments, as shown in FIG. 1, the system may be within the context of an ACO network 100 having one or more healthcare practices, referred to as Healthcare Practices (HP) 1 to n, which have access to a medical diagnosis identification application 160. Each practice (HP) can be a physician's office or a group practice having a plurality of practitioners, e.g., doctors, and possibly a plurality of different offices. Each practice (HP) in the ACO Network 100 can have its own EHR database system, e.g., EHR Systems (EHR) 1 to m. Typically, a practice will subscribe to a commercially available EHR system and have an account for remotely accessing its patient's EHRs. The practices access their respective EHRs to retrieve, review, and/or update electronic health records containing patient health information for each of its patients. Each EHR system may include a database management system and file server 110, 120 functionalities for electronically accessing, updating, and/or storing one or more health records in its storage devices 112, 122.

The ACO Network 100 may be implemented using one or more computing devices including servers, workstations, and networked computer systems comprising at least one processor, memory, and non-transitory computer-readable storage media storing executable instructions. The computer system of a healthcare practice that is part of the ACO Network 100 will have access to the medical diagnosis identification application 160, which can be accessible by users via a web browser 10, 20, or via an EHR overlay application rendered within a display, for example, disclosed in U.S. patent application Ser. No. 19/298,274 filed Aug. 13, 2025, entitled “Artificial Intelligence Overlay for Electronic Records . . . ” the contents of which are incorporated by reference in their entirety.” The ACO Network 100 may include network infrastructure such as routers, switches, and communication interfaces that facilitate data transmission between the healthcare practices, EHR systems, and the medical diagnosis identification application 160. In accordance with the inventive concepts, the medical diagnosis identification application 160 may also include an ML module 161, a verification module 162, and a diagnosis analyzer module 164.

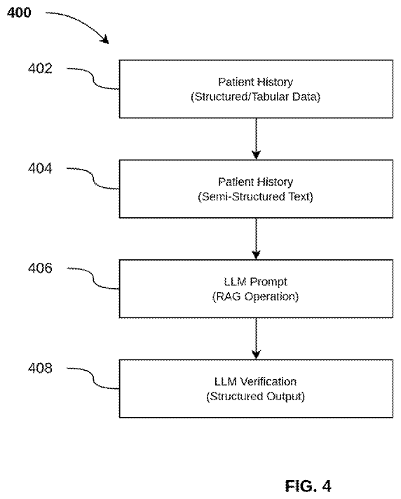

The application 160 is configured to receive input data from one or more sources, including but not limited to the EHR systems 1 to m. The ML module 161 receives and processes a set of structured data, for example, easy to parse electronic health records (EHRs). In some embodiments, the input data includes tabular structured patient data for diagnosis, for example shown in FIG. 4, arranged into rows and columns where the row includes a patient identifier, and the columns include health information such as age, vital information such as heartrate, medication, symptom, history insurance, and Dx suspects. The ML module 161 can perform an initial screening operation on the input data with high sensitivity to predict an initial broad set of suspect diagnoses. To achieve high sensitivity, the ML module 161 can apply a low decision threshold. For example, in a probability range of 0-1, a threshold can be reduced, for example, a model probability cutoff from 0.5 to 0.2 for a positive prediction to increase sensitivity, e.g., identify more positives. However, in doing so, more false positives may be identified because the precision decreases.

The verification module 163 can function as a “second opinion” on this data by filtering the predicted diagnoses generated by the ML module 161, and more specifically, clarifying the initial broad set of suspect diagnoses produced by the ML module 161. The verification module 163 includes a data converter 171 that is configured to convert the structured data into unstructured or semi-structured text for processing by an LLM prompt 173 of the verification module 162. LLMs are particularly good at working with unstructured data, for example, natural language text (e.g., emails, documents, reviews, social media), logs, images, and so on.

The diagnosis analyzer module 164 can generate diagnosis result data from the LLM prompt 173, which can be output to a computer for display to a medical practitioner or other user. For example, the diagnosis analyzer module 164 can aggregate the verified suspect diagnoses from the LLM prompt 173, associate each diagnosis with supporting clinical evidence and confidence levels, and format the results into a structured report that includes the identified HCC codes, relevant patient history excerpts, and recommended next steps for clinical review.

The diagnosis analyzer module 164 includes a fact-checking layer that cross-references each piece of LLM-generated evidence against the original source text. For each evidence “breadcrumb” produced by the LLM (e.g., “Patient has elevated BNP of 450 pg/mL on 2024 Mar. 15”), the fact-checker retrieves the source snippet from the episodic patient data and verifies that the claim has verbatim or near-verbatim support. The system uses string matching combined with semantic similarity scoring to identify supporting passages in the source material.

Evidence items that cannot be verified (similarity score below threshold or no matching source passage found) are flagged with a low-confidence indicator or removed entirely before presentation to clinicians. This prevents the LLM from hallucinating medical facts not present in the patient record. The fact-checking results are logged for model quality monitoring and can trigger alerts if hallucination rates exceed acceptable thresholds.

In some embodiments, the medical diagnosis identification application 160 includes an AI module 170 having an AI training module 172 configured to generate and train AI models, in particular, the ML module 161 and LLM module 162. In various embodiments, each AI model may be trained for a specific EHR system, EHR database type, and/or EHR database configuration. In various embodiments, the AI training module 172 may be used to train the models manually, automatically, or some combination thereof.

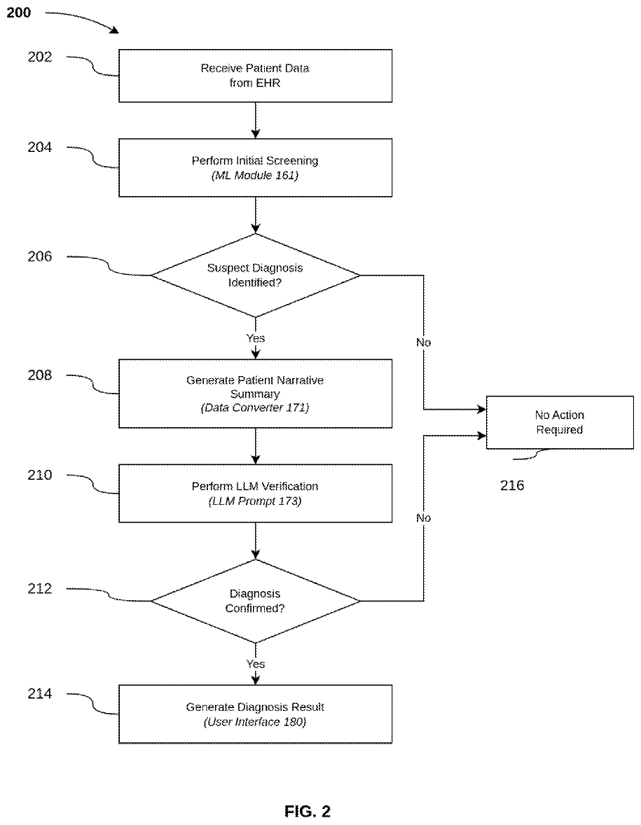

FIG. 2 is a flow diagram of a method 200 for potential diagnosis verification, in accordance with aspects of the inventive concepts. In describing the method 200, reference is made to elements of the system of FIG. 1.

The method 200 begins at an ML layer of the ML module 161, where patient data is received (202) from an EHR. The patient data may include structured EHR data such as demographic information, laboratory results, medication lists, diagnosis codes, vital signs, and claims data. In some embodiments, the patient data is received in a tabular format where each row corresponds to a patient identifier and each column contains specific health information attributes. The ML module 161 may access the patient data through electronic interfaces with one or more EHR systems (EHR 1 to m) within the ACO Network 100, retrieving relevant patient records for processing. The received patient data serves as the input for the initial screening operation performed by the high sensitivity ML model, which is configured to identify potential suspect diagnoses using low decision thresholds.

The ML module 161 performs an initial screening operation (204) on the received EHR patient data. As described above, the initial screening operation produces an initial broad set of suspect diagnoses due to the high sensitivity construction of the machine learning model(s) stored and executed by the ML module 161. In some embodiments, the initial screening operation applies the trained ML model to the structured patient data features to generate probability scores for each potential diagnosis category. The ML module 161 compares these probability scores against the low decision thresholds configured for high sensitivity operation. For example, if a probability score for a particular diagnosis exceeds the low threshold (e.g., 0.2 on a 0-1 scale), the diagnosis is flagged as a suspect diagnosis and included in the initial broad set. The high sensitivity configuration ensures that the ML module 161 captures a comprehensive range of potential diagnoses, including those with moderate confidence levels, thereby minimizing the risk of missing true positive cases. However, this approach may also result in the inclusion of false positives within the initial broad set, which are subsequently filtered by the verification module 162 in later stages of the method 200. In some embodiments, the ML module 161 may generate multiple suspect diagnoses for a single patient based on the patient's comprehensive health profile, including comorbidities and related conditions that may share overlapping clinical indicators.

At decision diamond 206, a determination is made whether one or more suspect diagnoses are produced from the initial screening operation. If no suspect diagnoses are identified, then no action is required (216). If one or more suspect diagnoses are identified by the ML module 162, then an LLM layer of the verification module 162 generates (208) a patient narrative unstructured summary from the EHR data (202). An LLM verification operation is performed (210) using the LLM prompt 173.

At decision diamond 212, a confirmation of the suspect diagnosis (206) is made. The verification module 162 evaluates the LLM-generated evidence and supporting rationale against the original patient record source text to determine whether sufficient clinical support exists for each suspect diagnosis. In some embodiments, the verification module 162 applies the hallucination fact-checking verification layer to cross-reference each piece of evidence, ensuring that evidence items have verbatim or near-verbatim support in the source data. The diagnosis analyzer module 164 may further assess whether the suspect diagnosis meets the criteria defined by clinical practice guidelines, evaluating the checklist-aligned fields including the criteria header, answer, source snippet, and confidence level. If a diagnosis is confirmed by the verification module 162 and diagnosis analyzer module 164, then the diagnosis result is generated (214) and output to a computer, for example, a user interface 180 for review by a medical practitioner. The diagnosis result may include the verified suspect diagnosis, associated HCC codes, supporting clinical evidence excerpts from the patient record, confidence levels, and any guideline-based recommendations for next steps if the evidence is incomplete. If the diagnosis is not confirmed, for example, because the LLM-generated evidence cannot be verified against the source text or because the clinical criteria are not sufficiently met, then no action is performed (216) and the suspect diagnosis is filtered from the output to reduce false positives and minimize clinician review burden.

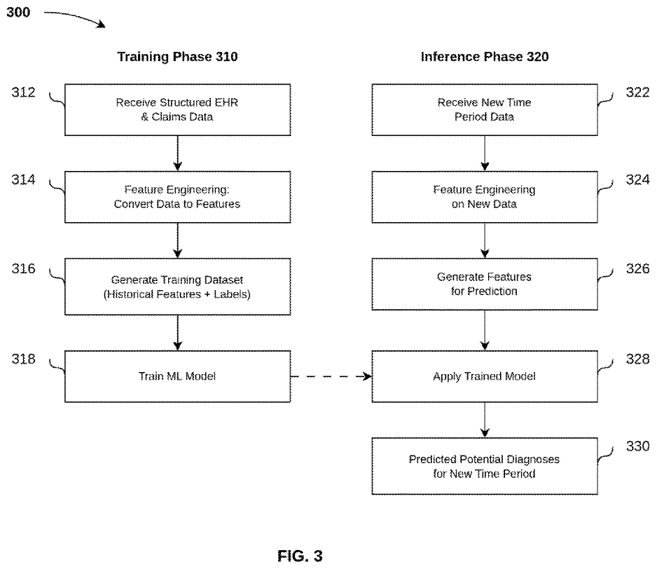

FIG. 3 is a flow diagram of a method 300 for ML model training and inference to predict potential diagnoses, in accordance with aspects of the inventive concepts. In describing the method 200, reference is made to elements of the system of FIG. 1. The method 300 can be categorized into a training phase 310 and an inference phase 320.

The training phase 310 may be performed by a combination of the ML module 161 and AI training module 172. In the training phase 310, structured EHR and claims data are received (312) by the ML module 161, for example, Other unstructured narratives like this may include a chief complaint, history of present illness, past medical history, medications, allergies, and vitals, lab results, diagnoses, test results, and so on in a tabular or other structured format.

At step 314, the tabular data is converted into features suitable for machine learning. Feature engineering may involve transforming raw patient data into numerical representations that capture clinically relevant patterns. This process may include encoding categorical variables (such as diagnosis codes or medication types), normalizing continuous variables (such as lab values or vital signs), creating temporal features (such as time since last encounter or frequency of visits), and aggregating related data points (such as combining multiple lab results into summary statistics). For example, a patient record containing a diagnosis code of “E11.9” (Type 2 diabetes), a hemoglobin A1c lab value of 8.2%, and a medication list including metformin may be converted into features such as: a binary indicator for diabetes diagnosis presence, a normalized A1c value relative to population ranges, a count of diabetes-related medications, and a time-based feature indicating days since the diabetes diagnosis was first recorded. These engineered features may then be used as input vectors for the machine learning model during training.

At step 316, a training dataset is generated from the features. In some embodiments, the training dataset includes a combination of historical features and labels. The labels may include known diagnoses from historical records such as COVID results, types of cancer, chronic conditions like diabetes or heart failure, and so on. The labels may be derived from confirmed diagnosis codes recorded in the EHR system, claims data indicating billed diagnoses, or clinician-verified outcomes from patient encounters. In some embodiments, the training dataset may be structured as a matrix where each row corresponds to a patient or patient encounter and each column corresponds to an engineered feature, with an additional column or set of columns containing the label or labels for that record. The historical records used to generate labels may span multiple time periods to capture longitudinal patient outcomes, and may include records from multiple healthcare practices within the ACO Network 100 to increase dataset diversity. In some aspects, the training dataset may undergo preprocessing steps such as handling missing values, balancing class distributions for rare diagnoses, and splitting into training, validation, and test subsets to facilitate model evaluation and prevent overfitting.

At step 318, the ML model 161 can be trained using the historical features and labels generated at step 316. In some embodiments, the training process may employ various machine learning algorithms such as gradient boosting machines (e.g., XGBoost, LightGBM), random forests, neural networks, or logistic regression models. The training process may involve iteratively adjusting model parameters to minimize a loss function that measures the difference between predicted and actual diagnoses. In some aspects, the optimization objective may be configured to prioritize sensitivity over specificity, for example, by using weighted loss functions that penalize false negatives more heavily than false positives. This configuration supports the high sensitivity operation of the ML module 161, ensuring that the model captures a broad set of potential diagnoses even at the cost of some false positives. The training process may also include hyperparameter tuning using techniques such as cross-validation, grid search, or Bayesian optimization, but not limited thereto, to identify optimal model configurations. In some embodiments, the decision threshold for classification may be set lower than the default (e.g., 0.2 instead of 0.5 on a probability scale of 0-1) to increase the model's sensitivity and ensure that fewer potential diagnoses are missed. The trained model may be validated against a test set to evaluate performance metrics including sensitivity, precision, and area under the receiver operating characteristic curve (AUC-ROC) before deployment.

The inference phase 320, or prediction, begins with the receipt of new time period data (322). The new time period data may include recently collected patient information such as new lab results, updated medication lists, recent vital sign measurements, newly recorded diagnosis codes, and claims data from recent encounters. In some embodiments, the new time period data may be received from one or more EHR systems within the ACO Network 100 on a scheduled basis, such as daily, weekly, or monthly, or may be triggered by specific events such as patient encounters or hospital admissions. At step 324, a pre-processing operation may be performed including feature engineering similar to step 314. This feature engineering process mirrors the training phase to ensure consistency between the features used during training and those used during inference. The pre-processing operation may include encoding categorical variables, normalizing continuous variables using the same scaling parameters established during training, creating temporal features, and aggregating related data points. Maintaining consistency between training and inference feature engineering helps ensure that the trained model can accurately interpret the new data. At step 326, features may be generated for prediction. The generated features may be organized into input vectors or matrices that match the format expected by the trained ML model, with each row corresponding to a patient and each column corresponding to an engineered feature.

At step 328, the trained ML model from step 318 can be applied to the generated features to produce predicted potential diagnoses (330) for the new time period. In some embodiments, the ML model generates probability scores for each potential diagnosis category for each patient. These probability scores may range from 0 to 1, indicating the model's confidence that a particular diagnosis applies to the patient. The ML module 161 compares each probability score against the low decision threshold configured for high sensitivity operation. For example, if the decision threshold is set to 0.2, any diagnosis with a probability score exceeding 0.2 may be flagged as a suspect diagnosis and included in the predicted potential diagnoses 330. In some aspects, the predicted potential diagnoses 330 may include multiple suspect diagnoses for a single patient, reflecting the patient's comprehensive health profile and potential comorbidities. The predicted potential diagnoses 330 may be output in a structured format that includes the patient identifier, the suspected diagnosis codes, and the associated probability scores. These predicted potential diagnoses 330 serve as the input for the subsequent LLM verification operation performed by the verification module 163, which filters and refines the broad set of suspect diagnoses to improve precision while maintaining the high sensitivity achieved by the ML model.

FIG. 4 is a flow diagram 400 of an LLM suspect verification operation, in accordance with aspects of the inventive concepts. Some or all of the illustrations in the flow diagram 400 can be generated by elements of the system of FIG. 1.

A set of structured table data (402) having a timestamp column is transformed, for example, by data converter 171 into a semi-structured format (404), namely, a patient history log, for input to an LLM, e.g., at LLM module 162. For example, a table may be used for training machine models. The table is created in which each record is a patient, and each column includes information about the patient. This initial format, namely, rows of structured event data, converted into narrative or timeline-style text information for each patient, is suitable for subsequent next-best-action-models, etc. performed by the LLM module 162, which can process the narrative or timeline-style text information for each patient arranged in a semi-structured format. The table can be converted into a machine learning feature set used for training the machine learning model, which can be used for ML predictions that are also derived from the tabular data. The predictions are used to refine them with LLM prompts.

In some embodiments, the LLM prompt 173 can perform a retrieval-augmented generation (RAG) operation (406) on the semi-structured data 404, e.g., patient histories, timelines, or clinical event logs, and generate (408) a result, for example, a set of predicted potential diagnoses refined from the patient history data. In some embodiments, the LLM prompt 173 shown in step 406 is used for predicting HCC codes from the structured/tabular data of step 404, such as diagnosis codes, demographics, as well as unstructured such as clinical notes.

In some embodiments, the LLM prompt 173 is structured using evidence-based clinical practice guidelines appropriate to the suspected diagnosis. For example, when validating a suspected CHF diagnosis, the prompt incorporates criteria from the American Heart Association/American College of Cardiology (AHA/ACC) guidelines for heart failure. The prompt instructs the LLM to evaluate the patient data against specific guideline criteria (e.g., “Does the patient have symptoms of volume overload?”, “Is there objective evidence of cardiac dysfunction?”).

The LLM output (408) is generated in a structured format with checklist-aligned “breadcrumbs” that correspond to guideline criteria. Each breadcrumb includes: (1) criteria_header identifying the guideline criterion being evaluated, (2) answer indicating whether the criterion is met (yes/no/partial), (3) type indicating the evidence category (e.g., “Laboratory Results”, “Clinical Symptoms”), (4) date of the evidence, (5) source_snippet containing verbatim text from the patient record, and (6) confidence_level (High/Medium/Low) indicating the LLM's certainty.

In embodiments where diagnosis criteria are not fully met (e.g., clinical symptoms present but confirmatory testing missing), the system generates a next_best_action recommendation derived from the same clinical guidelines. For example, if a patient presents with symptoms consistent with CHF but lacks recent BNP testing, the system may recommend “Order BNP or NT-proBNP test” as the next best action. Similarly, if diagnostic uncertainty remains after available testing, the system may recommend specialist referral (e.g., “Consider cardiology consultation”). These recommendations are presented alongside the suspect in the clinical user interface to guide physician workflow.

For example, a patient narrative may be derived from the unstructured EHR data (202) transformed into a semi-structure document (404) may pertain to a 58-year-old male with a history of congestive heart failure, kidney disease, and hypertension, presenting with lower extremity edema and weight gain over past week. The LLM prompt can process the semi-structured document (404), e.g., prompt: “Summarize this patient encounter into a structured medical summary” and produce an output (406). For example:

Patient: James Smith
Age: 58
Past Medical History:
Congestive Heart Failure (CHF)
Chronic Kidney Disease (Stage 3)
Hypertension
Chief Complaint: Lower extremity edema and weight gain
Duration: 1 week
Assessment: Likely CHF exacerbation
Plan:
Increase diuretic dose
Monitor weight and fluid intake

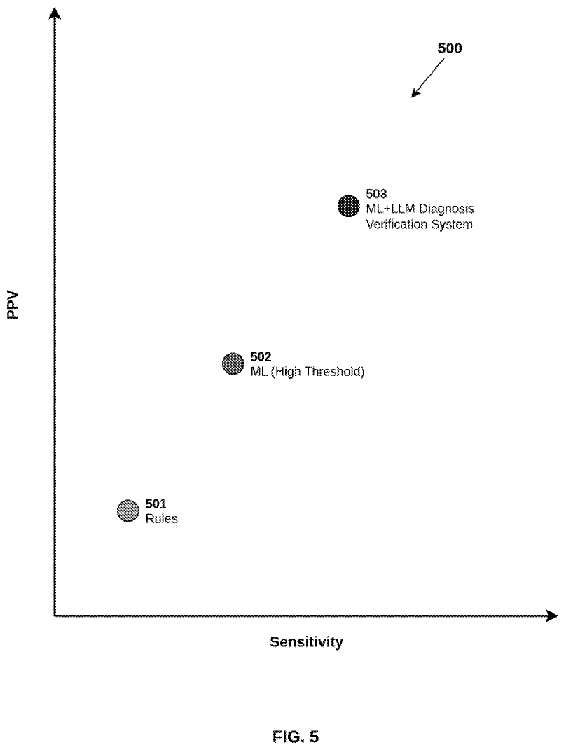

FIG. 5 is a graph 500 illustrating a quality-sensitivity comparison of techniques for capturing diagnosis (Dx) suspects, and more specifically, the trade-off between coverage and performance quality. Point 501 refers to a rule-based measurement. As shown, an issue with rules is low sensitivity and low quality. Heuristic rules have issues with respect to a limited coverage of suspects, and may have low sensitivity resulting in missed diagnoses, which is a problem when the goal is to identify as many true cases as possible, like in HCC coding. This can result in negative feedback from doctors.

Point 502 refers to an ML-only prediction. Although the ML-only result has a higher quality, measured in precision, and sensitivity than the rules-based result 501, the ML-only result 502 has a comparatively strict threshold, which limits coverage (measured in sensitivity) so that the ML model does not capture all nuances of the medical history input to the ML-model. The strict threshold set by the ML model may be used to set a high confidence requirement for the model to make a prediction. However, the ML model can only make predictions on a small subset of input, i.e., based on high confidence while labeling others as uncertain or unknown to reduce false positives or increase precision, which is often used in high-risk applications such as medical diagnoses.

Graphical point 503 refers to the result produced by the two-stage diagnosis verification system and method in the embodiments described in FIGS. 1-4, having both an increased sensitivity as compared to the rules-based (501) and ML only (502) results, but also providing greater coverage with respect to medical record history details or other medical record data and greater quality, i.e., fewer false positives.

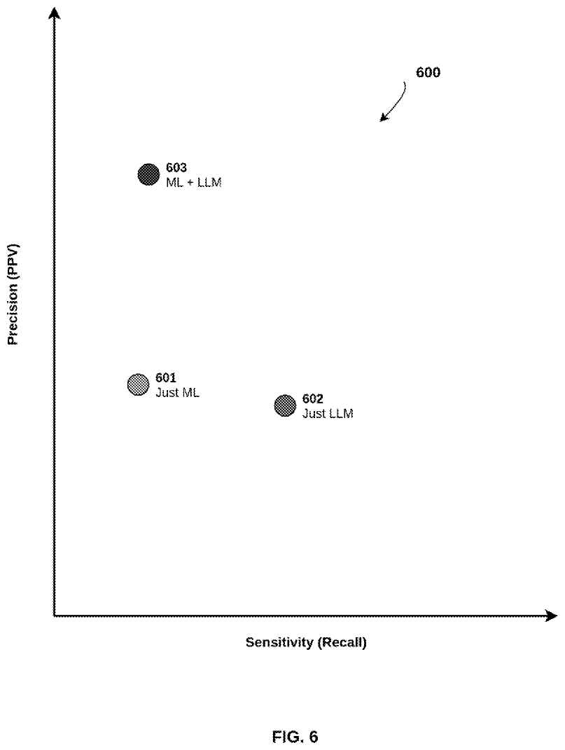

FIG. 6 is a graph 600 illustrating a tradeoff between the percentage of a total population that is marked, precision, and sensitivity of different models. As shown, an embodiment of the two-stage diagnosis verification system, which includes a combination of a high sensitivity ML model performed followed by an LLM performing a high precision analysis produces a result (point 603) that has a greater precision (PPV) than either an ML alone (601) or LLM alone (602), but with comparable sensitivity. More specifically, shown is the combination of the ML and LLM providing a desirable balance of the marked population, precision, and sensitivity. Point 603 corresponding to the ML/LLM combination indicates that fewer people are marked, which is less than that of an LLM-only application, which can reduce doctor fatigue with respect to the number of suspects. Although the number of suspects is less than the ML model only, the suspects under the ML/LLM combination are of higher quality, in particular, the precision is more detailed even at a comparable sensitivity.

Embodiments of the present inventive concept may address the challenge of retrieving relevant clinical evidence from heterogeneous medical data sources by providing a solution that retrieves and combines clinical evidence from both structured and unstructured sources. As mentioned above, the system may create AI-native data artifacts that effectively combine information from both structured EHR data such as lab results, medications, and diagnosis codes and unstructured clinical notes such as provider documentation and clinical narratives. By generating LLM-optimized semi-structured data, the system may provide complete context for AI models, bridging the gap between traditional structured data processing and unstructured text analysis. This approach enables the dual-stream retrieval architecture described below.

In some embodiments, the verification module 163 of the system described herein includes a dual-stream retrieval system that performs parallel semantic search over two distinct data sources. The first data source comprises structured patient data from a Patient Event Feed, including labs, medications, diagnoses, vitals, and procedures. The second data source comprises unstructured clinical notes from Continuity of Care Documents, including provider notes, assessment and plan sections, and history of present illness. Each stream uses an optimized query builder tailored to its data type; structured queries may use SQL-like filtering and aggregation, while unstructured queries may use natural language semantic search over embedded text. In some embodiments, the structured query builder may apply predicate-based filtering to identify relevant laboratory values, medication records, or diagnosis codes that match the suspected condition being evaluated. For example, when evaluating a suspected diagnosis of congestive heart failure, the structured query builder may retrieve BNP or NT-proBNP lab values, diuretic medications, and prior heart failure diagnosis codes. The unstructured query builder may employ vector embeddings to perform semantic similarity searches across clinical note text, identifying passages that discuss symptoms, clinical findings, or physician assessments relevant to the suspected diagnosis even when exact terminology varies across different providers or documentation styles.

Results from both streams may be deduplicated at the evidence-item level, removing redundant information that appears in both structured and unstructured forms, and combined into unified context. The deduplication process may identify when the same clinical fact appears in multiple sources, such as when a lab result is recorded in the structured EHR data and also mentioned in a provider's clinical note. In such cases, the system may retain a single representative evidence item while preserving links to all source documents. This context may be organized as episodic patient snippets, which are markdown-formatted summaries where each snippet represents a single clinical encounter such as a visit, hospitalization, or procedure. Within each snippet, structured and unstructured data may be temporally co-located by encounter date, providing the LLM with chronologically coherent clinical narratives rather than disconnected data points. This temporal organization may enable the LLM to understand the progression of a patient's condition over time, recognizing patterns such as worsening symptoms, response to treatment, or development of complications. The episodic snippets may include metadata linking back to source documents for subsequent fact-checking by the hallucination verification layer. In some embodiments, the metadata may include document identifiers, page numbers, section headers, and timestamps that enable precise retrieval of the original source text during the verification process.

In some embodiments, the system may include a hallucination fact-checking verification layer that cross-references LLM-generated evidence against original patient record source text. After the LLM generates evidence and supporting rationale for a potential diagnosis, each piece of evidence may be systematically verified by cross-referencing against the original patient record source text. The verification process may employ a combination of string matching algorithms and semantic similarity scoring to determine whether the LLM-generated evidence has adequate support in the source data. For string matching, the system may search for verbatim or near-verbatim occurrences of key terms, values, or phrases from the evidence claim within the source text. For semantic similarity scoring, the system may compute vector embeddings of both the evidence claim and candidate source passages, identifying passages with similarity scores above a configurable threshold. The system may ensure that every evidence item has verbatim or near-verbatim support in the source data, such as structured EHR data or unstructured clinical text. Evidence items that cannot be verified may be flagged with a low-confidence indicator or removed entirely before presentation to clinicians. In some embodiments, flagged evidence items may be presented to clinicians with a visual indicator distinguishing them from verified evidence, allowing clinicians to exercise additional scrutiny when reviewing unverified claims. This verification layer may help prevent medical data hallucination in high-stakes clinical applications. The fact-checking results may be logged for model quality monitoring, and the system may generate alerts if hallucination rates exceed acceptable thresholds, enabling continuous improvement of the LLM prompts and retrieval mechanisms.

In some embodiments, the LLM validation layer may use clinical practice guidelines to structure both the prompt and the expected output format. For example, guidelines from medical associations such as the American Heart Association, American Diabetes Association, or National Comprehensive Cancer Network may be used to define the criteria for diagnosis validation. The clinical practice guidelines may be encoded as structured criteria sets that specify the symptoms, laboratory findings, imaging results, or other clinical evidence required to support a particular diagnosis. The LLM prompt may instruct the model to evaluate the patient data against each criterion in the guideline, generating a structured assessment of whether the evidence supports the suspected diagnosis. The LLM output may be organized as checklist-aligned fields including a criteria header derived from the guideline, an answer indicating whether the criteria is met (such as yes, no, or partial), an evidence type indicating the category of supporting evidence (such as laboratory results, clinical symptoms, or imaging findings), a date indicating when the evidence was recorded, a source snippet providing verbatim proof from the patient record, and a confidence level indicating the LLM's certainty in the assessment (such as high, medium, or low). This approach may ensure that AI reasoning follows established medical criteria and provides clinicians with transparent, auditable justification for each diagnostic recommendation.

In some embodiments, when the LLM validation identifies incomplete evidence for a suspected diagnosis, the system may generate specific guideline-based recommendations for next steps. These recommendations may include specific lab tests to order, diagnostic procedures to perform, or specialist referrals. For example, if a patient presents with symptoms consistent with diabetes but lacks recent hemoglobin A1c testing, the system may recommend ordering an A1c test. If a patient has elevated cardiac biomarkers but no recent echocardiogram, the system may recommend an echocardiogram to assess cardiac function. If diagnostic uncertainty remains after available testing, the system may recommend specialist consultation, such as cardiology for suspected heart failure or nephrology for suspected chronic kidney disease. The recommendations may be derived from the same clinical guidelines used for diagnosis validation, ensuring consistency between the diagnostic criteria and the recommended next steps. This functionality may extend the platform from diagnostic identification to clinical workflow integration, providing actionable guidance to clinicians when diagnosis criteria are not fully met. In some embodiments, the recommendations may be prioritized based on clinical urgency, cost-effectiveness, or availability, enabling clinicians to select the most appropriate next step for each patient's circumstances.

Systems and methods in accordance with the inventive concepts represent a transformative step in enhancing the EHR experience for physicians. By focusing on robust data handling, advanced AI model development, seamless system integration, and a user-centric approach, systems and methods in accordance with the inventive concepts deliver significant improvements in healthcare delivery and physician satisfaction.

While the foregoing has described what are considered to be the best mode and/or other preferred embodiments, it is understood that various modifications can be made therein and that the inventive concepts may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the inventive concepts, which are, for clarity, described in the context of separate embodiments, may also be provided in combination in a single embodiment. Conversely, various features of the invention which are, for brevity, described in the context of a single embodiment may also be provided separately or in any suitable sub-combination.

For example, it will be appreciated that all of the features set out in any of the claims (whether independent or dependent) can be combined in any given way.

What is claimed is:

1. A method for identifying a medical diagnosis, comprising:
receiving, by a first model, structured data, wherein the first model includes a Machine Learning (ML) model;
generating, by the first model, a first set of potential diagnoses
converting the structured data into a set of semi-structured data; and
processing, by a second model, the set of semi-structured data to generate a second set of potential diagnoses that is a subset of the first set of potential diagnoses; wherein:
the second model includes a Large Language Model (LLM); and
the second set of potential diagnoses is smaller than the first set of potential diagnoses.

2. The method of claim 1, further comprising:
applying a first feature engineering operation to the structured data; and
training the ML model.

3. The method of claim 2, further comprising:
predicting, by the LLM, potential diagnoses for a new time period, comprising:
applying a second feature engineering operation on new time period data; and
applying the trained ML model to features generated by the second feature engineering operation.

4. The method of claim 1, further comprising:
executing the LLM prompt independently of the results of the ML model.

5. The method of claim 1, wherein processing, by the LLM, the set of semi-structured data includes predicting Hierarchical Condition Category (HCC) codes from the structured data.

6. The method of claim 1, wherein the LLM performs a verification operation on the set of potential diagnoses to identify a subset set of potential diagnoses with better precision.

7. The method of claim 6, wherein the LLM prompt is constructed and arranged to perform a retrieval-augmented generation (RAG) operation on the semi-structured data.

8. The method of claim 1, wherein the ML model performs an initial screening of one or more suspect diagnoses from the structured data and the LLM performs a refined screening of the one or more suspect diagnoses to determine whether the one or more suspect diagnoses are to be output to a user computer.

9. The method of claim 1, wherein the structured data includes electronic health records (EHR) in a tabular format.

10. A method for diagnosis code identification, comprising:
receiving, by a first model including a Machine Learning (ML) model, a plurality of diagnosis codes in a structured format and grouped into categories that represent serious or chronic conditions;
generating, by the ML model, a prediction of a first set of diagnosis codes for a patient;
converting the diagnosis codes structured format into a set of semi-structured data; and
processing, by a second model including an LLM, the set of semi-structured data to determine a second set of diagnosis codes from the diagnosis codes.

11. The method of claim 10, wherein the diagnosis codes include HCC diagnosis codes.

12. A system for identifying and verifying a potential medical diagnosis, comprising:
at least one processor and at least one computer storage device;
a high sensitivity machine learning model stored at the at least one computer storage device and executable by the at least one processor to:
electronically access at least one electronic health records (EHR) system comprising a plurality of patient EHRs; and
identify a first set of suspect diagnoses using low decision thresholds;
a converter to convert structured EHR data including the plurality of patient EHRs and the first set of suspect diagnoses into a cohesive unstructured patient narrative; and
a LLM stored at the at least one computer storage device and executable by the at least one processor to identify a second set of potential diagnoses from the first set of suspect diagnoses.

13. A method for clinical reasoning using parallel semantic search, comprising:
performing parallel retrieval from two distinct data streams comprising structured patient data and unstructured clinical notes, wherein the structured patient data includes labs, medications, and diagnoses, and the unstructured clinical notes include provider documentation;
using separate query builders optimized for each data type;
deduplicating and combining results from both data streams into unified context; and
organizing the unified context as episodic snippets, wherein the episodic snippets comprise encounter-level patient summaries that temporally organize both structured and unstructured data by clinical episode.

14. A method for verifying LLM-generated medical evidence, comprising:
generating, by an LLM, evidence and supporting rationale for a potential medical diagnosis;
systematically verifying each piece of evidence by cross-referencing against original patient record source text;
determining whether each evidence item has verbatim or near-verbatim support in the source text; and
flagging or removing evidence items that cannot be verified before presentation to clinicians.

15. The method of claim 6, wherein:
the LLM uses clinical practice guidelines to structure both a prompt and an expected output format; and
the LLM output is organized as checklist-aligned fields including a criteria header from the clinical practice guidelines, an answer, a source snippet as verbatim proof, and a confidence level.

16. The method of claim 15, further comprising:
when the LLM identifies incomplete evidence for a suspected diagnosis, generating guideline-based recommendations for next steps, wherein the recommendations include at least one of: specific lab tests to order, diagnostic procedures to perform, or specialist referrals; and
wherein the recommendations are derived from the same clinical practice guidelines used for diagnosis validation.
